(19) United States
(12) Patent Application Publication
(10) Pub. No.: US 2005/0246003 A1
(43) Pub. Date: Nov. 3, 2005

(54) Stimulation lead having pairs of stimulating electrodes spaced at different distances for providing electrical stimulation to different nerve tissues

(75) Inventors: Damon R. Black, Dallas, TX (US); Tracy L. Cameron, Toronto (CA)

Correspondence Address: BAKER BOTTS L.L.P., 2001 ROSS AVENUE, 6TH FLOOR, DALLAS, TX, 75201-2980, US

(73) Assignee: Advanced Neuromodulation Systems, Inc.

(21) Appl. No.: 11/116,483

(22) Filed: Apr. 28, 2005

Related U.S. Application Data
(60) Provisional application No. 60/566,308, filed on Apr. 28, 2004.

(51) Int. Cl. A61N 1/05

(52) U.S. Cl. 607/116

(57) ABSTRACT

According to one embodiment, an electrical stimulation lead adapted for implantation in the body provides therapeutic electrical stimulation of nerve tissue in the body. A first stimulating electrode, a second stimulating electrode, and a third stimulating electrode are integrated into the electrical stimulation lead. The second stimulating electrode is located between the first and third stimulating electrodes. The electrical stimulation lead provides electrical stimulation to either first nerve tissue in the body or second nerve tissue in the body according to whether the first and second stimulating electrodes or the first and third stimulating electrodes are activated. The first and second stimulating electrodes may be activated to deliver electrical stimulation pulses to the first nerve tissue. The first and third stimulating electrodes, but not the second stimulating electrode, may be activated to deliver electrical stimulation pulses to the second nerve tissue.

### RELATED APPLICATIONS

This application claims the benefit under 35 U.S.C. § 119(e) of U.S. Provisional Application Ser. No. 60/566,308, filed Apr. 28, 2004.

### TECHNICAL FIELD OF THE INVENTION

This invention relates generally to electrical stimulation of nerve tissue in a person's body and in particular to a stimulation lead having stimulating electrodes spaced at different distances for providing electrical stimulation to different nerve tissues.

### BACKGROUND

Many people experience adverse conditions associated with functions of the cortex, the thalamus, and other brain structures. Such conditions have been treated effectively by delivering electrical energy to one or more target areas of the brain. One method of delivering electrical energy to the brain involves inserting an electrical stimulation lead including multiple stimulating electrodes through a burr hole formed in a person's skull and then positioning the lead in a precise location such that one or more of the stimulating electrodes are positioned proximate target nerve tissue in the person's brain correlated to a condition in the person's body. One or more pairs of the stimulating electrodes may deliver electrical stimulation pulses to the target nerve tissue to treat the condition in the person's body. For example, particular nerve tissue in the brain may be stimulated to treat tremor from a movement disorder such as Parkinson's Disease. A variety of other clinical conditions may also be treated with deep brain stimulation, such as essential tremor, tremor from multiple sclerosis or brain injury, or dystonia or other movement disorders.

The electrical stimulation lead implanted in the brain is connected to an implantable pulse generator implanted at a separate site in the body, such as in the upper chest. The implantable pulse generator generates electrical stimulation pulses that are delivered to the target nerve tissue by the stimulating electrodes. According to one technique, a set of efficacious stimulation parameters are determined and entered into the implantable pulse generator. Once implanted, the pulse generator transmits electrical stimulation pulses to the stimulating electrodes of the implanted stimulation lead according to the set of parameters, and the stimulating electrodes deliver the electrical stimulation pulses to the target nerve tissue in the brain.

Different stimulation leads may be used for stimulating different target nerve tissue, such as target nerve tissue of different sizes, different types of target nerve tissue, or target nerve tissue in different locations in the brain, for example. In particular, different stimulation leads may have different spacing between pairs of stimulating electrodes. Thus, for stimulation of particular nerve tissue, a stimulation lead may be selected having stimulating electrodes that are appropriately spaced for providing electrical stimulation to the particular nerve tissue. For example, a stimulation lead having stimulating electrodes spaced relatively close together may be used for stimulating nerve tissue in the subthalamic nucleus (STN) to treat cardinal symptoms of Parkinson's Disease, while a different stimulation lead having stimulating electrodes spaced further apart may be used for stimulating nerve tissue in the thalamus (such as nerve tissue in the ventro-intermediate thalamus (VIM) or in the Globus Pallidus Internus (Gpi), for example) to treat tremor due to Parkinson's Disease or essential tremor.

Electrical stimulation leads may also be used to apply electrical stimulation to nerve tissue in the spinal cord or a peripheral nerve to treat regions of the body affected by chronic pain from a variety of etiologies. As discussed above with regard to stimulation of nerve tissue in the brain, an implantable pulse generator may transmit electrical stimulation pulses to the implanted electrical stimulation lead according to a preprogrammed set of parameters and, in response, the stimulating electrodes of the implanted stimulation lead may deliver the electrical stimulation pulses to the target nerve tissue in the spinal cord or a peripheral nerve. In some instances, the electrical stimulation pulses stimulate the target nerve tissue in the spinal cord or a peripheral nerve to cause a subjective sensation of numbness or tingling in the affected region of the body, known as “paresthesia,” which masks or otherwise relieves pain in the affected region. For example, the stimulating electrodes may be located external to the dura adjacent particular nerve tissue in the spinal cord that is to be stimulated.

As discussed above with regard to stimulation of nerve tissue in the brain, different stimulation leads may be used for stimulating different target nerve tissue in the spinal cord or a peripheral nerve, such as target nerve tissue of different sizes, different types of target nerve tissue, or target nerve tissue in different locations in the person's body, for example. In particular, different stimulation leads may have different spacing between pairs of stimulating electrodes for stimulating different nerve tissues in the spinal cord or a peripheral nerve. Thus, a stimulation lead having appropriately-spaced stimulating electrodes may be selected based on the particular nerve tissue in the spinal cord or peripheral nerve to be stimulated.

### SUMMARY OF THE INVENTION

The electrical stimulation system, lead, and method of the present invention may reduce or eliminate certain problems and disadvantages associated with previous techniques for stimulating nerve tissue to treat conditions in the body.

According to one embodiment, an electrical stimulation lead adapted for implantation in the body provides therapeutic electrical stimulation of nerve tissue in the body. The electrical stimulation lead includes a first stimulating electrode, a second stimulating electrode, and a third stimulating electrode integrated into the electrical stimulation lead. The second stimulating electrode is located between the first and third stimulating electrodes. The first and second stimulating electrodes are operable to cooperate to deliver electrical stimulation pulses to first nerve tissue in the body. The first and third stimulating electrodes are operable to cooperate to deliver electrical stimulation pulses to second nerve tissue in the body. The electrical stimulation lead is operable to provide electrical stimulation to either the first nerve tissue or the second nerve tissue according to whether the first and second stimulating electrodes or the first and third stimulating electrodes are activated. The first and second stimulating electrodes are activated to provide electrical stimulation to the first nerve tissue, while the first and third stimulating electrodes, but not the second stimulating electrode, are activated to provide electrical stimulation to the second nerve tissue.

Particular embodiments of the present invention may provide one or more technical advantages. According to the present invention, an electrical stimulation lead adapted for implantation in a person's body includes multiple stimulating electrodes that may be activated or deactivated such that different pairs of stimulating electrodes having different spacing therebetween may be used to stimulate different nerve tissues in the person's body. For example, in certain embodiments, a first pair of stimulating electrodes spaced apart from each other by a first distance along an electrical stimulation lead may be activated to provide electrical stimulation pulses to first nerve tissue in a person's body, while a second pair of stimulating electrodes spaced apart from each other by a second distance along the electrical stimulation lead may be activated to provide electrical stimulation pulses to second nerve tissue in a person's body. A particular stimulating electrode may be used in both the first and the second pairs of stimulating electrodes. The stimulating electrodes on a single stimulation lead may be activated and deactivated as desired to provide differently-spaced pairs of activated stimulating electrodes to apply electrical stimulation pulses to different nerve tissues. As a result, a single stimulation lead may be used for stimulating different nerve tissues, thereby reducing or eliminating the need for multiple stimulation leads having stimulating electrodes spaced at different distances for stimulating different nerve tissues. Thus, the number of stimulation leads needed to be manufactured, distributed, and kept in inventory for stimulating various nerve tissues in the body may be significantly reduced.

Certain embodiments may provide all, some, or none of these advantages. Certain embodiments may provide one or more other advantages, one or more of which may be apparent to those skilled in the art from the figures, descriptions, and claims included herein.

### BRIEF DESCRIPTION OF THE DRAWINGS

For a more complete understanding of the present invention and advantages thereof, reference is now made to the following description taken in conjunction with the accompanying drawings, in which:

FIGS. 1A-1B illustrate example electrical stimulation systems 10 for electrically stimulating various nerve tissue in a person's body;

FIGS. 2A-2I illustrate example electrical stimulation leads 14 that may be used for electrically stimulating nerve tissue in a person's body to treat a condition in the person's body;

FIG. 3 illustrates an example arrangement of stimulating electrodes along a percutaneous stimulation lead;

FIG. 4 illustrates an example of a person undergoing placement of an electrical stimulation lead for stimulation of nerve tissue in the person's brain using stereotactic equipment to guide lead placement and a lead-securing apparatus to secure stimulation lead in position in the person's brain;

FIG. 5 illustrates an example stimulation set;

FIG. 6 illustrates a number of example stimulation programs, each of which includes a number of stimulation sets;

FIG. 7 illustrates example execution of a sequence of stimulation sets within an example stimulation program; and

FIG. 8 illustrates an example method of programming and implanting the stimulation system 10 of FIGS. 1A-1B into a person's brain in order to electrically stimulate nerve tissue in the person's brain to treat a condition in the person's body.

### DESCRIPTION OF EXAMPLE EMBODIMENTS

According to the present invention, an electrical stimulation lead adapted for implantation in a person's body includes multiple stimulating electrodes that may be activated or deactivated such that different pairs of stimulating electrodes having different spacing therebetween may be used to stimulate different nerve tissues in the person's body. In certain embodiments, a first pair of stimulating electrodes spaced apart from each other by a first distance along an electrical stimulation lead may be activated to provide electrical stimulation pulses to first nerve tissue in a person's body, while a second pair of stimulating electrodes spaced apart from each other by a second distance along the electrical stimulation lead may be activated to provide electrical stimulation pulses to second nerve tissue in a person's body. A particular stimulating electrode may be used in both the first and the second pairs of stimulating electrodes. For example, in an electrical stimulation lead including a number of stimulating electrodes, a first electrode may be activated along with (a) a second stimulating electrode separated from the first electrode by a first distance such that the first and second stimulating electrodes may cooperate to deliver electrical stimulation pulses to the first nerve tissue, or (b) a third stimulating electrode separated from the first electrode by a second distance greater than the first distance such that the first and third stimulating electrodes may cooperate to deliver electrical stimulation pulses to the second nerve tissue. The second stimulating electrode may be located between the first and third electrodes. In certain embodiments, the second distance (between the first and third stimulating electrodes) is approximately an even multiple of the first distance (between the first and second stimulating electrodes), for example, three times the first distance.

Thus, on a particular stimulation lead, different pairs of stimulating electrodes having different spacing therebetween may be activated to stimulate different nerve tissues in a person's body. Such different nerve tissues may include, for example, nerve tissues of different sizes, different types of nerve tissue, or nerve tissue in different locations in the person's body. For example, a first pair of stimulating electrodes having a first spacing therebetween may be used to provide electrical stimulation to nerve tissue in the subthalamic nucleus (STN), while a second pair of stimulating electrodes (which may include one of the stimulating electrodes of the first pair) having a second spacing therebetween greater than the first spacing may be used to provide electrical stimulation to nerve tissue in the thalamus, such as nerve tissue in the ventro-intermediate thalamus (VIM) or Globus Pallidus Internus (Gpi).

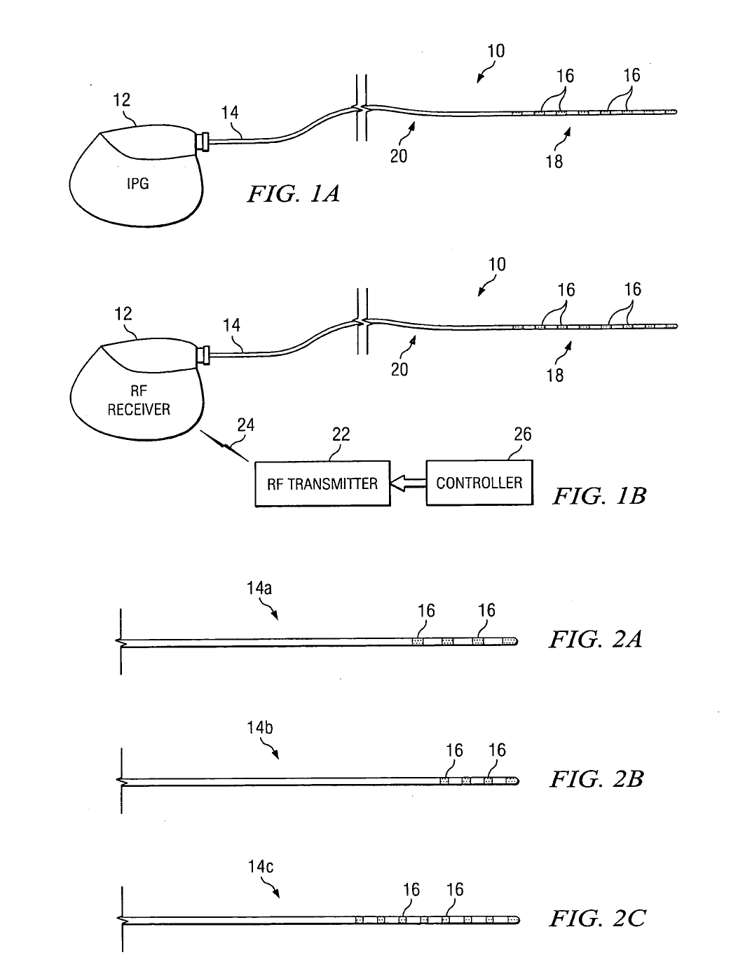

FIGS. 1A-1B illustrate example electrical stimulation systems 10 for electrically stimulating various nerve tissue in a person's body. In general terms, stimulation system 10 includes an implantable electrical stimulation source 12 for generating electrical stimulation pulses and an implantable electrical stimulation lead 14 having a plurality of stimulating electrodes 16 integrated into a stimulating portion 18 of electrical stimulation lead 14. Stimulating electrodes 16 are operable to apply the electrical stimulation pulses generated by electrical stimulation source 12 to nerve tissue in the body to treat a condition in the body correlated to the nerve tissue. Each of the stimulating electrodes 16 may be activated or deactivated such that different pairs of stimulating electrodes 16 having different spacing therebetween may be used to stimulate different nerve tissues in the body. For example, a first pair of stimulating electrodes 16 having a first spacing therebetween may be activated to deliver electrical stimulation pulses to first nerve tissue in the body to treat a particular condition in the body correlated to the first nerve tissue, while a second pair of stimulating electrodes 16 having a second spacing therebetween different from the first spacing may be activated to deliver electrical stimulation pulses to second nerve tissue in the body to treat a condition in the body correlated to the second nerve tissue.

In operation, the electrical stimulation source 12 and electrical stimulation lead 14 are implanted in the person's body, as discussed below with reference to FIG. 4. Stimulation source 12 is coupled to a connecting portion 20 of electrical stimulation lead 14. In certain embodiments, stimulation source 12 controls the electrical stimulation pulses transmitted to one or more pairs of activated stimulating electrodes 16 located in, on, near, or otherwise proximate the nerve tissue, according to suitable stimulation parameters, including whether particular stimulating electrodes 16 are activated or deactivated, as well as various parameters (such as the duration, amplitude (or intensity), frequency, etc.) of stimulation pulses that activated stimulating electrodes 16 deliver to the nerve tissue. In certain embodiments, a doctor, the patient, or another user of stimulation source 12 may directly or indirectly input various stimulation parameters to specify or modify the nature of the electrical stimulation provided.

In one embodiment, as shown in FIG. 1A, stimulation source 12 includes an implantable pulse generator (IPG). An example IPG may be one manufactured by Advanced Neuromodulation Systems, Inc., such as the Genesis® System, part numbers 3604, 3608, 3609, and 3644. In another embodiment, as shown in FIG. 1B, stimulation source 12 includes an implantable wireless receiver. An example wireless receiver may be one manufactured by Advanced Neuromodulation Systems, Inc., such as the Renew® System, part numbers 3408 and 3416. The wireless receiver is capable of receiving wireless signals from a wireless transmitter 22 located external to the person's body. The wireless signals are represented in FIG. 1B by wireless link symbol 24. A doctor, the patient, or another user of stimulation source 12 may use a controller 26 located external to the person's body to provide control signals for operation of stimulation source 12. Controller 26 provides the control signals to wireless transmitter 22, wireless transmitter 22 transmits the control signals and power to the wireless receiver of stimulation source 12, and stimulation source 12 uses the control signals to vary the stimulation parameters of electrical stimulation pulses transmitted through electrical stimulation lead 14 to the stimulation site. An example wireless transmitter 122 may be one manufactured by Advanced Neuromodulation Systems, Inc., such as the Renew® System, part numbers 3508 and 3516.

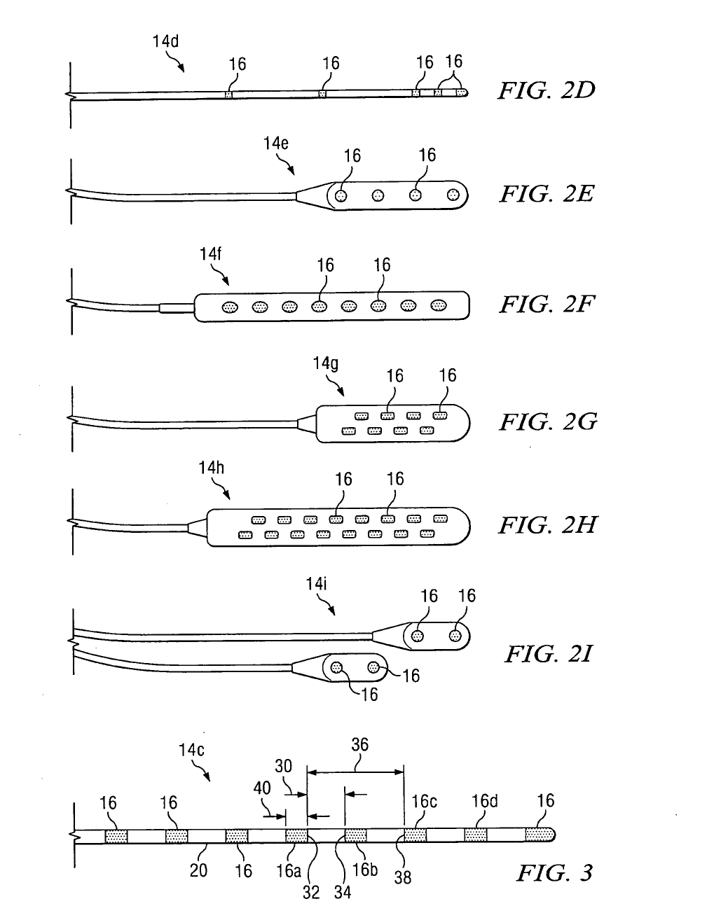

FIGS. 2A-2I illustrate example electrical stimulation leads 14 that may be used for electrically stimulating nerve tissue in a person's body to treat a condition in the person's body. As described above, each of the one or more stimulation leads 14 incorporated in stimulation system 10 includes one or more stimulating electrodes 16 adapted to be positioned in, on, near, or otherwise proximate nerve tissue in a person's body such that one or more pairs of stimulating electrodes 16 may be activated to deliver to the nerve tissue electrical stimulation pulses received from stimulation source 12 to treat a condition in the person's body correlated to the nerve tissue. As discussed above, different pairs of stimulating electrodes 16 having different spacing therebetween may be activated to deliver electrical stimulation pulses to different nerve tissues, such as nerve tissues of different sizes, different types of nerve tissue, or nerve tissue in different locations in the body, for example.

A percutaneous stimulation lead 14, such as example leads 14a-d, includes one or more circumferential stimulating electrodes 16 spaced apart from one another along the length of stimulation lead 14. Circumferential stimulating electrodes 16 emit electrical stimulation energy generally radially in all directions. A laminotomy or paddle stimulation lead 14, such as example leads 14e-i, includes one or more directional stimulating electrodes 16 spaced apart from one another along one surface of stimulation lead 14. Directional stimulating electrodes 16 emit electrical stimulation energy in a direction generally perpendicular to the surface of stimulation lead 14 on which they are located. Although various types of stimulation leads 14 are shown as examples, the present invention contemplates stimulation system 10 including any suitable type of stimulation lead 14 in any suitable number. In addition, a stimulation lead 14 may be used alone or in combination with one or more other stimulation leads 14. For example, unilateral stimulation of nerve tissue in the brain is typically accomplished using a single stimulation lead 14 implanted in one side of the brain, while bilateral stimulation of the brain is typically accomplished using two leads 14 implanted in opposite sides of the brain.

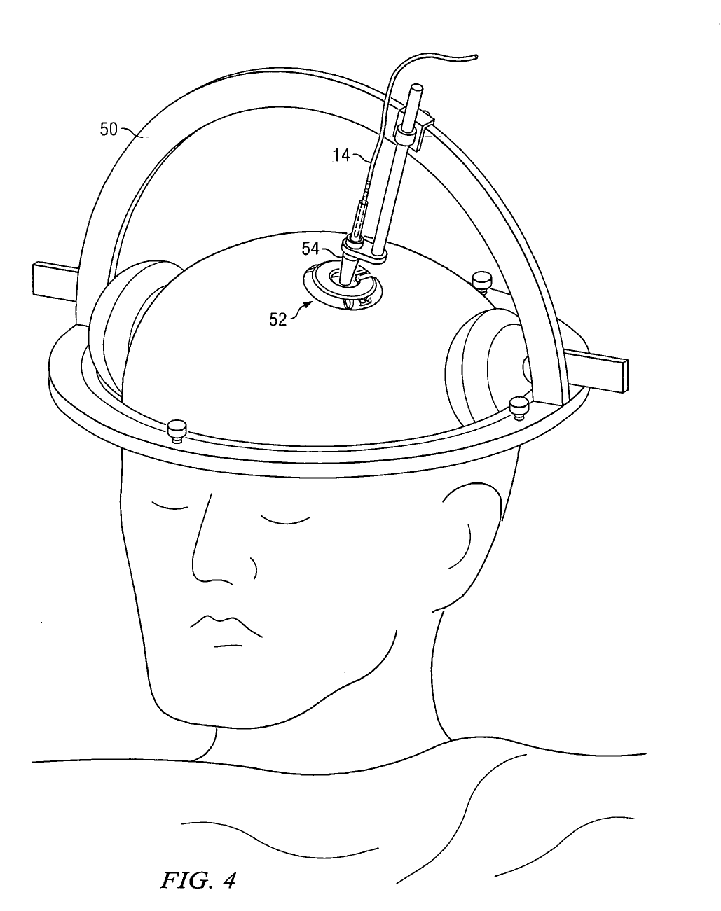

FIG. 3 illustrates an example arrangement of stimulating electrodes 16 along example percutaneous stimulation lead 14c shown in FIG. 2C. A plurality of stimulating electrodes 16 are spaced along stimulating portion 18 of stimulation lead 14c. In certain embodiments, more than four stimulating electrodes 16 may be evenly spaced along stimulating portion 18. In the particular embodiment shown in FIG. 3, eight stimulating electrodes 16 are evenly spaced along stimulating portion 18.

Pairs of adjacent stimulating electrodes 16 are separated from one another by a distance 30 extending between the nearest edges of the adjacent stimulating electrodes 16. For example, the distance 30 between adjacent stimulating electrodes 16a and 16b extends from a first edge 32 of stimulating electrode 16a facing stimulating electrode 16b to a first edge 34 of stimulating electrode 16a facing stimulating electrode 16a. Pairs of non-adjacent stimulating electrodes 16, which are pairs of stimulating electrodes 16 having another stimulating electrode 16 located therebetween, are separated by a distance 36 extending between the nearest edges of the adjacent stimulating electrodes 16. For example, the distance 36 between non-adjacent stimulating electrodes 16a and 16c extends from the first edge 32 of stimulating electrode 16a to a first edge 38 of stimulating electrode 16c facing stimulating electrode 16a.

In certain embodiments, the distance 30 between adjacent stimulating electrodes 16 is less than 1.5 mm. In a particular embodiment, the distance 30 between adjacent stimulating electrodes 16 is approximately 0.5 mm. In certain embodiments, the distance 36 between non-adjacent stimulating electrodes 16 is between approximately 1.0 mm and approximately 2.0 mm. In a particular embodiment, the distance 36 between non-adjacent stimulating electrodes 16 is approximately 1.5 mm. In addition, in certain embodiments, the distance 36 between non-adjacent stimulating electrodes 16 is approximately an even multiple of the distance 30 between adjacent stimulating electrodes 16, such as a multiple of two, three, or four, for example. To illustrate, in a particular embodiment the distance 30 between adjacent stimulating electrodes 16 is approximately 0.5 mm and the distance 36 between non-adjacent stimulating electrodes 16 is approximately 1.5 mm, or approximately three times the distance 30 between adjacent stimulating electrodes 16.

Stimulating electrodes 16 of various sizes may be used in various stimulation leads 14. In certain embodiments, the length of stimulating electrodes 16 along stimulation lead 14c, indicated as length 40, may be less than 1.5 mm. In a particular embodiment, the length 40 of stimulating electrodes 16 is approximately 0.5 mm. Stimulating electrodes 16 may be formed from any one or more materials suitable for forming an electrode. For example, in certain embodiments, stimulating electrodes 16 are formed from platinum iridium.

As discussed above, one or more different pairs of stimulating electrodes 16 on stimulation lead 14c may be activated to provide stimulation to different nerve tissues such as, for example, nerve tissues of different sizes, different types of nerve tissue, or nerve tissue in different locations in the person's body, to treat various conditions correlated to such nerve tissue. In particular, differently-spaced pairs of stimulating electrodes 16 on stimulation lead 14c may be activated for providing stimulation to different nerve tissues. The stimulating electrodes 16 on a single stimulation lead 14c may be activated and deactivated as desired to provide differently-spaced pairs of activated stimulating electrodes 16 to apply electrical stimulation pulses to different nerve tissues. Thus, a single stimulation lead 14c may be used for stimulating different nerve tissues, thereby reducing or eliminating the need for multiple stimulation leads having stimulating electrodes spaced at different distances from each other. As a result, the number of stimulation leads needed to be manufactured, distributed, and kept in inventory for stimulating various nerve tissues in the body may be reduced.

For example, one or more pairs of adjacent stimulating electrodes 16 (such as stimulating electrodes 16a and 16b or stimulating electrodes 16b and 16c, for example) or one or more pairs of non-adjacent stimulating electrodes 16 (such as stimulating electrodes 16a and 16c, stimulating electrodes 16b and 16d, or stimulating electrodes 16a and 16d, for example) may be activated to provide stimulation to different nerve tissues. As used herein, “a pair of adjacent stimulating electrodes” or “an adjacent pair of stimulating electrodes” refers to any pair of stimulating electrodes 16 having no stimulating electrodes 16 located therebetween. In contrast, as used herein, “a pair of non-adjacent stimulating electrodes” or “a non-adjacent pair of stimulating electrodes” refers to any pair of stimulating electrodes 16 having one or more stimulating electrodes 16 located therebetween.

As an example of using different pairs of stimulating electrodes 16 to stimulate different nerve tissues, one or more pairs of adjacent stimulating electrodes 16 may be used to provide electrical stimulation to nerve tissue in the subthalamic nucleus (STN) to treat cardinal symptoms of Parkinson's Disease, while one or more pairs of non-adjacent stimulating electrodes 16 may be used for stimulating nerve tissue in the thalamus (such as nerve tissue in the ventro-intermediate thalamus (VIM) or in the Globus Pallidus Internus (Gpi), for example) to treat tremor due to Parkinson's Disease or essential tremor. In a particular embodiment, one or more pairs of adjacent stimulating electrodes 16 having a distance 30 of approximately 0.5 mm may be used to provide electrical stimulation to nerve tissue in the subthalamic nucleus (STN), while one or more non-adjacent stimulating electrodes 16 having a distance 36 of approximately 1.5 mm may be used for stimulating nerve tissue in the thalamus. As discussed above, a particular stimulating electrode 16 may be used both as part of a pair of adjacent stimulating electrodes 16 and as part of a pair of non-adjacent stimulating electrodes 16, depending on which other stimulating electrodes 16 are activated or deactivated. Thus, for example, stimulating electrode 16a may be activated along with (a) stimulating electrode 16b to form a pair of adjacent stimulating electrodes 16 for delivering electrical stimulation pulses to a first nerve tissue, or (b) stimulating electrode 16c to form a pair of non-adjacent stimulating electrodes 16 for delivering electrical stimulation pulses to a second nerve tissue.

It should be understood that the various parameters discussed above regarding stimulating electrodes 16 on stimulation lead 14c, such as the spacing, length and composition of stimulating electrodes 16, may similarly apply to stimulating electrodes 16 on any other suitable stimulation lead 14, including the stimulating electrodes 16 on each of the stimulation leads 14a-14b shown in FIGS. 2A-2B and stimulation leads 14d-14i shown in FIGS. 2D-2I.

FIG. 4 illustrates an example of a person undergoing placement of an electrical stimulation lead 14 for stimulation of nerve tissue in the brain using stereotactic equipment 50 to guide lead placement and a lead-securing apparatus 52 to secure stimulation lead 14 in position in the person's brain. As can be appreciated from FIG. 4, electrical stimulation lead 14 is typically coupled to stereotactic equipment 50 during lead placement for increased stability and housed within an insertion cannula 54 for insertion into the brain. Stereotactic equipment 50, lead-securing apparatus 52, and cannula 54 cooperate to guide the insertion of stimulation lead 14 into the person's brain through a burr hole formed in the person's skull.

In certain embodiments, electrical stimulation lead 14 is positioned within the brain such that one or more pairs of stimulating electrodes 16 are located in, on, near, or otherwise proximate nerve tissue 54 within one or more particular regions 58 of the brain, for example, particular regions of the frontal lobe, the occipital lobe, the parietal lobe, the temporal lobe, the cerebellum, or the brain stem. As an example, stimulation lead 14 may be positioned such that: (a) a first pair of adjacent stimulating electrodes 16 spaced apart from each other by a first distance 30 are located in, on, near, or otherwise proximate nerve tissue in the subthalamic nucleus (STN) and activated to treat, for example, cardinal symptoms of Parkinson's Disease; or (b) a second pair of non-adjacent stimulating electrodes 16 (which may share a stimulating electrode 16 with the first pair of stimulating electrodes 16) spaced apart from each other by a second distance 36 greater than the first distance 30 are located in, on, near, or otherwise proximate nerve tissue in the thalamus (such as nerve tissue in the ventro-intermediate thalamus (VIM) or in the Globus Pallidus Internus (Gpi), for example) and activated to treat, for example, tremor due to Parkinson's Disease or essential tremor. However, it should be understood that stimulating electrodes 16 may be located in, on, near, or otherwise proximate any nerve tissue in any region of the brain or any other location in the body to treat various conditions correlated to such nerve tissue.

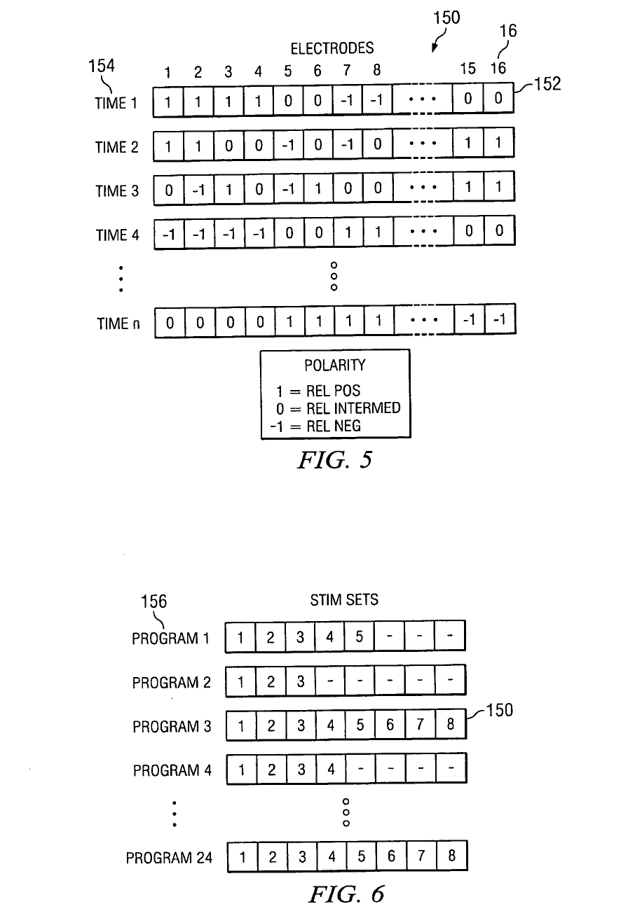

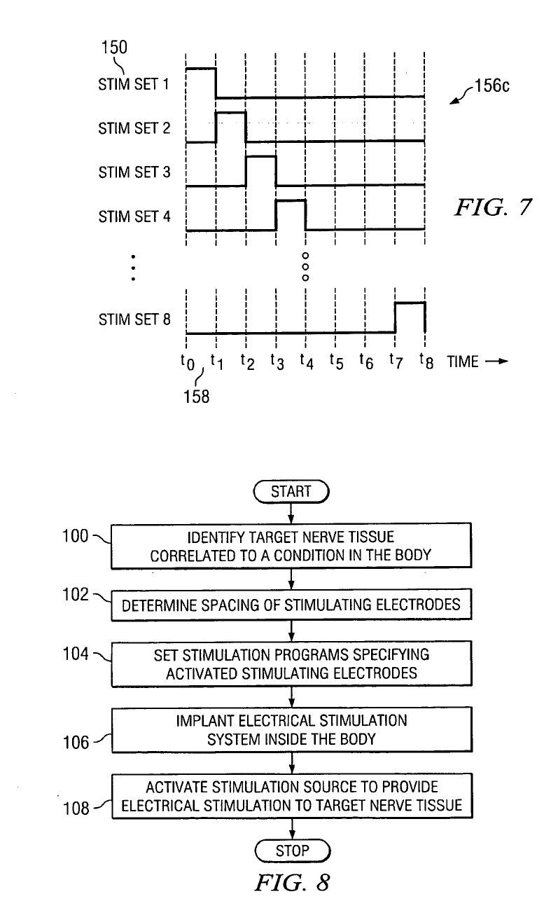

FIG. 5 illustrates an example stimulation set 150. One or more stimulation sets 150 may be provided, each stimulation set 150 specifying a number of stimulation parameters for the stimulation set 150. For example, as described more fully below with reference to FIGS. 6-7, multiple stimulation sets 150 may be executed in a suitable sequence according to a pre-programmed or randomized stimulation program. Example stimulation parameters for a stimulation set 150 may define whether particular stimulating electrodes 16 are activated or deactivated, as well as an amplitude (or intensity), a frequency, phase information, and a pulse width for each of a series of stimulation pulses that activated stimulating electrodes 16 are to deliver to the target nerve tissue during a time interval during which stimulation set 150 is executed, along with a polarity 152 for each stimulating electrode 16 within each stimulation pulse. In general, in particular embodiments in which electrical stimulation lead 14 includes two or more stimulating electrodes 16, electric fields are generated between adjacent stimulating electrodes 16 having different polarities 152 to deliver electrical stimulation pulses to the target nerve tissue. In particular embodiments in which electrical stimulation lead 14 includes a single stimulating electrode 16, such as a single stimulating electrode 16 at the tip of stimulation lead 14 for example, electric fields are generated between the single stimulating electrode 16 and a terminal or other electrical contact associated with stimulation source 12. Stimulation parameters may also include a pulse shape, for example, biphasic cathode first, biphasic anode first, or any other suitable pulse shape.

The polarity for a stimulating electrode 16 at a time 154 beginning a corresponding stimulation pulse or sub-interval within a stimulation pulse may be a relatively positive polarity 152, a relatively negative polarity 152, or an intermediate polarity 152 between the relatively positive polarity 152 and relatively negative polarity 152. For example, the relatively positive polarity 152 may involve a positive voltage, the relatively negative polarity 152 may involve a negative voltage, and the relatively intermediate polarity 152 may involve a zero voltage (i.e. “high impedance”). As another example, the relatively positive polarity 152 may involve a first negative voltage, the relatively negative polarity 152 may involve a second negative voltage more negative than the first negative voltage, and the relatively intermediate polarity 152 may involve a negative voltage between the first and second negative voltages. The availability of three distinct polarities 152 for an stimulating electrode 16 may be referred to as “tri-state” electrode operation. The polarity 152 for each stimulating electrode 16 may change for each of the sequence of times 154 corresponding to stimulation pulses or to sub-intervals within a stimulation pulse according to the stimulation parameters specified for the stimulation set 150. For example, as is illustrated in FIG. 5 for an example stimulation set 150 for a stimulation lead 14 with sixteen stimulating electrodes 16, the polarities 152 of the sixteen stimulating electrodes 16 may change for each of the sequence of times 154. In the example of FIG. 5, a relatively positive polarity 152 is represented using a “1,” a relatively intermediate polarity 152 is represented using a “0,” and a relatively negative polarity 152 is represented using a “−1,” although any values or other representations may be used.

FIG. 6 illustrates a number of example stimulation programs 156, each including a number of stimulation sets 150. One or more stimulation programs 156 may be set up to provide electrical stimulation of target nerve tissue. As described above, each stimulation set 150 specifies a number of stimulation parameters for the stimulation set 150. In one embodiment, within each stimulation program 156, stimulation system 16 consecutively executes the sequence of one or more stimulation sets 150 associated with stimulation program 156. The sequence may be executed only once, repeated a specified number of times, or repeated an unspecified number of times within a specified time period. For example, as is illustrated in FIG. 7 for the third example stimulation program 156c including eight stimulation sets 150, each of the eight stimulation sets 150 is consecutively executed in sequence. Although the time intervals 158 (t₁-t₀, t₂-t₁, etc.) during which the stimulation sets 150 are executed are shown as being equal, the present invention contemplates a particular stimulation set 150 being executed over a different time interval 158 than one or more other stimulation sets 150 according to particular needs.

Although stimulation system 16 is illustrated for example as accommodating up to twenty-four stimulation programs 156 each including up to eight stimulation sets 150, the present invention contemplates any number of stimulation programs 156 each including any number of stimulation sets 150. For example, in a very simple case, a single stimulation program 156 may include a single stimulation set 150, whereas in a more complex case twenty-four stimulation programs 156 may each include eight stimulation sets 150.

In one embodiment, stimulation system 16 executes only a single stimulation program 156 in response to user selection of that stimulation program for execution. In another embodiment, during a stimulation period, stimulation system 16 executes a sequence of pre-programmed stimulation programs 156 for each stimulation lead 14 until the stimulation period ends. Depending on the length of the stimulation period and the time required to execute a sequence of stimulation programs 156, the sequence may be executed one or more times. For example, the stimulation period may be defined in terms of a predetermined number of cycles each involving a single execution of the sequence of stimulation programs 156, the sequence of stimulation programs 156 being executed until the predetermined number of cycles has been completed. As another example, the stimulation period may be defined in terms of time, the sequence of stimulation programs 156 being executed until a predetermined time interval has elapsed or the patient or another user manually ends the stimulation period. Although a sequence of stimulation programs 156 is described, a single stimulation program being executed one or more times during a stimulation period according to particular needs. Furthermore, the present invention contemplates each stimulation program 156 being executed substantially immediately after execution of a previous stimulation program 156 or after a suitable time interval has elapsed since the completion of the previous stimulation program 156.

Where stimulation system 16 includes multiple stimulation leads 14, stimulation programs 156 for one stimulation lead 14 may be executed substantially simultaneously as stimulation programs 156 for one or more other stimulation leads 14, may be alternated with stimulation programs 156 for one or more other stimulation leads 14, or may be arranged in any other suitable manner with respect to stimulation programs 156 for one or more other stimulation leads 14.

In general, each stimulation program 156 may, but need not necessarily, be set up for electrical stimulation of different nerve tissues. As an example, for electrical stimulation of the brain, one or more stimulation programs 156 may be set up for therapeutic electrical stimulation of certain nerve tissue in the brain and one or more other stimulation programs 156 may be set up for electrical stimulation certain other nerve tissue in the brain.

The present invention contemplates any suitable circuitry within stimulation source 12 for generating and transmitting signals for electrical stimulation of target nerve tissue within a person's body. Example circuitry that may be suitable for use is illustrated and described in U.S. Patent 6,609,031 B1, which is hereby incorporated by reference herein as if fully illustrated and described herein.

FIG. 8 illustrates an example method of programming and implanting the stimulation system 10 of FIGS. 1A-1B into a person's brain in order to electrically stimulate nerve tissue in the person's brain to treat a condition in the person's body. At step 100, target nerve tissue in a person's brain correlated to a condition in the person's body is identified. In some embodiments, a portion of the person's brain is imaged using one or more imaging techniques to identify the target nerve tissue in the person's brain correlated to the condition in the person's body. In other embodiments, the location of the target nerve tissue in the person's brain correlated to the condition in the person's body is determined using imaging studies performed on other patients suffering from the same or similar condition as the person.

At step 102, a desired distance 30, 36 between stimulating electrodes 16 to be activated for stimulating the target nerve tissue is determined based at least on the target nerve tissue identified at step 100. For example, the desired distance 30, 36 between stimulating electrodes 16 to be activated may be determined based on the size of the target nerve tissue, the type of the target nerve tissue, the location of the target nerve tissue in the brain, or any combination of the preceding.

At step 104, one or more stimulation programs 156, each including one or more stimulation sets 150, may be programmed into stimulation system 10 for providing electrical stimulation to the target nerve tissue identified at step 100. Stimulation programs 156 specify whether each stimulating electrode 16 is activated or deactivated based on the desired distance 30,36 between activated stimulating electrodes 16 determined at step 104. For example, if the desired distance 30,36 between activated stimulating electrodes 16 determined at step 104 is relatively small, one or more pairs of adjacent stimulating electrodes 16 may be programmed to be activated. In contrast, if the desired distance 30,36 between activated stimulating electrodes 16 determined at step 104 is relatively large, one or more pairs of non-adjacent stimulating electrodes 16 may be programmed to be activated. Thus, stimulation programs 156 may be programmed into stimulation system 10 to provide differently-spaced pairs of activated stimulating electrodes 16 on single stimulation lead 14, which may reduce or eliminate the need to select from multiple stimulation leads, as discussed above. In addition to specifying which stimulating electrodes 16 are activated or deactivated, stimulation programs 156 may also specify various parameters (such as the duration, amplitude (or intensity), frequency, etc.) of stimulation pulses that the activated stimulating electrodes 16 are to deliver to the target nerve tissue in the brain.

Electrical stimulation system 10 is implanted inside the person at step 106. First, the skull is prepared by exposing the skull and creating a burr hole in the skull. Lead-securing apparatus 52 may be fixed to the scalp or skull using sutures, screws, or other suitable fixators. Stereotactic equipment 50 suitable to aid in placement of electrical stimulation lead 14 in the brain may be positioned around the head. Insertion cannula 54 for electrical stimulation lead 14 is inserted into the brain. For example, a hollow needle may provide cannula 54. Cannula 54 and electrical stimulation lead 14 may be inserted together or stimulation lead 14 may be inserted through cannula 54 after cannula 54 has been inserted. Using stereotactic imaging guidance or otherwise, electrical stimulation lead 14 is then precisely positioned within the brain such that one or more pairs of stimulating electrodes 16 on stimulation lead 14 are located in, on, near, or otherwise proximate the target nerve tissue in the brain correlated to the condition in the person's body.

In certain embodiments, imaging information of nerve tissue in the brain is downloaded into a neuronavigation system that is used to precisely position stimulation lead 14 within the brain. Such imaging information may be obtained by imaging the person's brain using any suitable imaging technique (such as, for example, positron emission tomography (PET), magnetic resonance imaging (MRI), functional MRI (fMRI), single photon emission computed tomography (SPECT), transcranial magnetic stimulation (TMS), and optical imaging) or using imaging studies of other patients suffering from the same or similar condition as the person.

Once electrical stimulation lead 14 has been positioned in the brain and secured using lead-securing apparatus 52, stimulation lead 14 is uncoupled from any stereotactic equipment 50, and cannula 54 and any stereotactic equipment 50 are removed. Where stereotactic equipment 50 is used, cannula 54 may be removed before, during, or after removal of stereotactic equipment 50. Connecting portion 20 of electrical stimulation lead 14 is laid substantially flat along the skull. Once electrical stimulation lead 14 has been inserted and secured, stimulation lead 14 extends from the lead insertion site to the implant site at which stimulation source 12 is implanted. The implant site is typically a subcutaneous pocket formed to receive and house stimulation source 12. The implant site is usually positioned a distance away from the insertion site, such as near the buttocks or another place in the torso area.

At step 108, stimulation source 12 is activated, which generates and sends electrical stimulation pulses via activated stimulating electrodes 16 to the target nerve tissue in the brain according to the one or more stimulation programs 156 programmed at step 106. As discussed above, the one or more stimulation programs 156 programmed at step 106 may specify which stimulating electrodes 16 are activated to provide one or more pairs of activated stimulating electrodes 16 having the desired spacing therebetween. In certain embodiments, the electrical stimulation pulses delivered to the target nerve tissue by the activated stimulating electrodes 16 may adjust the activity of the target nerve tissue in an appropriate manner to treat the condition in the person's body.

Although example steps are illustrated and described, the present invention contemplates two or more steps taking place substantially simultaneously or in a different order. In addition, the present invention contemplates using methods with additional steps, fewer steps, or different steps, so long as the steps remain appropriate for determining the desired distance 30, 36 for one or more pairs of activated stimulating electrodes 16 on a stimulation lead 14 for stimulating target nerve tissue, controlling whether particular stimulating electrodes 16 are activated or deactivated to provide one or more pairs of active stimulating electrodes 16 having the determined spacing, and implanting the stimulation lead 14 into a person for electrical stimulation of the target nerve tissue to treat a condition in the person's body correlated to the target nerve tissue.

Although the present invention has been described with several embodiments, a number of changes, substitutions, variations, alterations, and modifications may be suggested to one skilled in the art, and it is intended that the invention encompass all such changes, substitutions, variations, alterations, and modifications as fall within the spirit and scope of the appended claims.

1. An electrical stimulation lead adapted for implantation in the body to provide therapeutic electrical stimulation of nerve tissue in the body, comprising:
a first stimulating electrode integrated into the electrical stimulation lead;
a second stimulating electrode integrated into the electrical stimulation lead and operable to cooperate with the first stimulating electrode to deliver electrical stimulation pulses to first nerve tissue in the body; and
a third stimulating electrode integrated into the electrical stimulation lead and positioned such that the second stimulating electrode is located between the first and third stimulating electrodes, the third stimulating electrode operable to cooperate with the first stimulating electrode to deliver electrical stimulation pulses to second nerve tissue in the body;
the electrical stimulation lead being operable to provide electrical stimulation to either the first nerve tissue or the second nerve tissue according to whether the first and second stimulating electrodes or the first and third stimulating electrodes are activated:
the first and second stimulating electrodes being activated to provide electrical stimulation to the first nerve tissue; and
the first and third stimulating electrodes being activated and the second stimulating electrode not being activated to provide electrical stimulation to the second nerve tissue.

2. The electrical stimulation lead of claim 1, wherein the first nerve tissue comprises nerve tissue in the subthalamic nucleus (STN) and the second nerve tissue comprises nerve tissue in the thalamus.

3. The electrical stimulation lead of claim 2, wherein the second nerve tissue comprises nerve tissue in the ventro-intermediate thalamus (VIM) or the Globus Pallidus Internus (Gpi).

4. The electrical stimulation lead of claim 1, wherein:
the electrical stimulation lead extends generally in a first direction; and
the length of the second stimulating electrode in the first direction is less than or approximately equal to 1.0 mm.

5. The electrical stimulation lead of claim 1, wherein:
a first distance extends from an edge of the first stimulating electrode facing the second stimulating electrode to an edge of the second stimulating electrode facing the first stimulating electrode;
a second distance extends from the edge of the first stimulating electrode facing the second stimulating electrode to an edge of the third stimulating electrode facing the first stimulating electrode; and
the second distance is approximately an even multiple of a first distance

6. The electrical stimulation lead of claim 1, wherein:
a first distance extending from an edge of the first stimulating electrode facing the second stimulating electrode to an edge of the second stimulating electrode facing the first stimulating electrode is between approximately 0.25 mm and approximately 1.0 mm; and
a second distance extending from the edge of the first stimulating electrode facing the second stimulating electrode to an edge of the third stimulating electrode facing the first stimulating electrode is between approximately 1.0 mm and approximately 2.0 mm.

7. The electrical stimulation lead of claim 1, wherein:
the second and third stimulating electrodes are operable to cooperate to deliver electrical stimulation pulses to the first nerve tissue; and
the electrical stimulation lead is operable to provide electrical stimulation to either the first nerve tissue or the second nerve tissue according to whether the first and second stimulating electrodes, the second and third stimulating electrodes, or the first and third stimulating electrodes are activated:
the first and second stimulating electrodes, the second and third stimulating electrodes, or both being activated to provide electrical stimulation to the first nerve tissue; and
the first and third stimulating electrodes being activated and the second stimulating electrode not being activated to provide electrical stimulation to the second nerve tissue.

8. The electrical stimulation lead of claim 1, wherein:
the electrical stimulation lead comprises a plurality of stimulating electrodes integrated into the electrical stimulation lead and positioned in a row, the plurality of stimulating electrodes including the first, second, and third stimulating electrodes;
each adjacent pair of stimulating electrodes of the plurality of stimulating electrodes are separated from each other by a first distance and operable to cooperate with each other to deliver electrical stimulation pulses to the first nerve tissue, each adjacent pair of stimulating electrodes comprising a pair of stimulating electrodes having no other stimulating electrodes located therebetween;
each non-adjacent pair of stimulating electrodes of the plurality of stimulating electrodes are separated from each other by a second distance greater than the first distance and operable to cooperate with each other to deliver electrical stimulation pulses to the second nerve tissue, each non-adjacent pair of stimulating electrodes comprising a pair of stimulating electrodes having one or more other stimulating electrodes located therebetween; and
the electrical stimulation lead is operable to provide electrical stimulation to either the first nerve tissue or the second nerve tissue according to whether an adjacent pair of stimulating electrodes or a non-adjacent pair of stimulating electrodes are activated:
an adjacent pair of stimulating electrodes being activated to provide electrical stimulation to the first nerve tissue; and
a non-adjacent pair of stimulating electrodes being activated and the one or more other stimulating electrodes located therebetween not being activated to provide electrical stimulation to the second nerve tissue.

9. The electrical stimulation lead of claim 8, wherein the electrical stimulation lead comprises at least five stimulating electrodes.

10. The electrical stimulation lead of claim 1, wherein the electrical stimulation lead is a percutaneous lead.

11. A stimulation system for providing therapeutic electrical stimulation to nerve tissue in the body, comprising:
a stimulation source adapted for implantation in the body and operable to generate and transmit electrical stimulation pulses; and
an electrical stimulation lead coupled to the stimulation source and adapted for implantation in the body, the electrical stimulation lead comprising:
a first stimulating electrode integrated into the electrical stimulation lead;
a second stimulating electrode integrated into the electrical stimulation lead and operable to cooperate with the first stimulating electrode to deliver the electrical stimulation pulses to first nerve tissue in the body; and
a third stimulating electrode integrated into the electrical stimulation lead and positioned such that the second stimulating electrode is located between the first and third stimulating electrodes, the third stimulating electrode operable to cooperate with the first stimulating electrode to deliver the electrical stimulation pulses to second nerve tissue in the body;
the electrical stimulation lead being operable to deliver the electrical stimulation pulses to either the first nerve tissue or the second nerve tissue according to whether the first and second stimulating electrodes or the first and third stimulating electrodes are activated:
the first and second stimulating electrodes being activated to deliver the electrical stimulation pulses to the first nerve tissue; and
the first and third stimulating electrodes being activated and the second stimulating electrode not being activated to deliver the electrical stimulation pulses to the second nerve tissue.

12. The system of claim 11, wherein the first nerve tissue comprises nerve tissue in the subthalamic nucleus (STN) and the second nerve tissue in the body comprises nerve tissue in the thalamus.

13. The system of claim 12, wherein the second nerve tissue comprises nerve tissue in the ventro-intermediate thalamus (VIM) or the Globus Pallidus Internus (Gpi).

14. The system of claim 11, wherein:
the electrical stimulation lead extends generally in a first direction; and
the length of the second stimulating electrode in the first direction is less than or approximately equal to 1.0 mm.

15. The system of claim 11, wherein:
a first distance extends from an edge of the first stimulating electrode facing the second stimulating electrode to an edge of the second stimulating electrode facing the first stimulating electrode;
a second distance extends from the edge of the first stimulating electrode facing the second stimulating electrode to an edge of the third stimulating electrode facing the first stimulating electrode; and
the second distance is approximately an even multiple of a first distance

16. The system of claim 11, wherein:
a first distance extending from an edge of the first stimulating electrode facing the second stimulating electrode to an edge of the second stimulating electrode facing the first stimulating electrode is between approximately 0.25 mm and approximately 1.0 mm; and
a second distance extending from the edge of the first stimulating electrode facing the second stimulating electrode to an edge of the third stimulating electrode facing the first stimulating electrode is between approximately 1.0 mm and approximately 2.0 mm.

17. The system of claim 11, wherein:
the second and third stimulating electrodes are operable to cooperate to deliver electrical stimulation pulses to the first nerve tissue; and
the electrical stimulation lead is operable to provide electrical stimulation to either the first nerve tissue or the second nerve tissue according to whether the first and second stimulating electrodes, the second and third stimulating electrodes, or the first and third stimulating electrodes are activated:
the first and second stimulating electrodes, the second and third the second stimulating electrodes, or both being activated to provide electrical stimulation to the first nerve tissue; and
the first and third stimulating electrodes being activated and the second stimulating electrode not being activated to provide electrical stimulation to the second nerve tissue.

18. A stimulation system for providing therapeutic electrical stimulation to nerve tissue in the body, comprising:
a stimulation source adapted for implantation in the body and operable to generate and transmit electrical stimulation pulses; and
an electrical stimulation lead coupled to the stimulation source and adapted for implantation in the body, the electrical stimulation lead comprising:
a first stimulating electrode integrated into the electrical stimulation lead;
a second stimulating electrode integrated into the electrical stimulation lead and operable to cooperate with the first stimulating electrode to deliver the electrical stimulation pulses to first nerve tissue in the body; and
a third stimulating electrode integrated into the electrical stimulation lead and positioned such that the second stimulating electrode is located between the first and third stimulating electrodes, the third stimulating electrode operable to cooperate with the first stimulating electrode to deliver the electrical stimulation pulses to second nerve tissue in the body;
each adjacent pair of stimulating electrodes of the plurality of stimulating electrodes are separated from each other by a first distance and operable to cooperate with each other to deliver electrical stimulation pulses to the first nerve tissue, each adjacent pair of stimulating electrodes comprising a pair of stimulating electrodes having no other stimulating electrodes located therebetween;
each non-adjacent pair of stimulating electrodes of the plurality of stimulating electrodes are separated from each other by a second distance greater than the first distance and operable to cooperate with each other to deliver electrical stimulation pulses to the second nerve tissue, each non-adjacent pair of stimulating electrodes comprising a pair of stimulating electrodes having one or more other stimulating electrodes located therebetween; and
the electrical stimulation lead being operable to deliver the electrical stimulation pulses to either the first nerve tissue or the second nerve tissue according to whether an adjacent pair of stimulating electrodes or a non-adjacent pair of stimulating electrodes are activated:
an adjacent pair of stimulating electrodes being activated to provide electrical stimulation to the first nerve tissue; and
a non-adjacent pair of stimulating electrodes being activated and the one or more other stimulating electrodes located therebetween not being activated to provide electrical stimulation to the second nerve tissue.

19. The system of claim 18, wherein the electrical stimulation lead comprises at least five stimulating electrodes.

20. The system of claim 11, wherein the electrical stimulation lead is a percutaneous lead.
